(19) United States
(12) Patent Application Publication
(10) Pub. No.: US 2010/0120049 A1
(43) Pub. Date: May 13, 2010

(54) BIOMARKERS FOR SERIOUS SKIN RASH

(76) Inventors: Aris FLORATOS, Astoria, NY (US); Sally L. JOHN, New London, CT (US); Matthew R. NELSON, Chapel Hill, NC (US)

Correspondence Address: WILMERHALE/DC, 1875 PENNSYLVANIA AVE., NW, WASHINGTON, DC, 20006, US

(21) Appl. No.: 12/615,944

(22) Filed: Nov. 10, 2009

Related U.S. Application Data
(60) Provisional application No. 61/112,983, filed on Nov. 10, 2008.
(60) Provisional application No. 61/168,875, filed on Apr. 13, 2009.

(51) Int. Cl. C12Q 1/68

(52) U.S. Cl. 435/6

(57) ABSTRACT

The present invention provides a method for predicting the risk of a patient for developing adverse drug reactions, particularly Serious Skin Rash (SSR), including such severe adverse reactions such as Stevens-Johnson Syndrome (SJS) and Toxic Epidermal Necrolysis (TEN). The invention also provides a method of identifying a subject afflicted with or at risk of developing SSR. In some aspects, the methods comprise analyzing at least one genetic marker, wherein the presence of the at least one genetic marker indicates that the subject is afflicted with or at risk of developing SSR. Genetic markers useful in accordance with the methods of the invention are disclosed.

### RELATED APPLICATIONS

This application claims priority under 35 USC §119 to U.S. Provisional Application No. 61/112,983 filed Nov. 10, 2008, and U.S. Provisional Application No. 61/168,875 filed Apr. 13, 2009, the disclosures of which are incorporated by reference herein in their entireties.

### BACKGROUND

Adverse reactions to drugs are a major cause of morbidity and death. Frequently occurring adverse drug reactions include cutaneous reactions. Although drug eruptions may range from mild to moderate, such as maculopapular rash, erythema multiforme (EM), urticaria, and fixed drug eruption, more severe adverse reactions, such as Stevens-Johnson Syndrome (SJS) and Toxic Epidermal Necrolysis (TEN), are life-threatening and frequently result in death.

SJS and TEN are characterized by similar presentations, with TEN being more severe and having a higher mortality rate. These presentations include acute exanthema, which progresses towards limited (SJS) or widespread (TEN) blistering and erosion of the skin and mucous membranes.

Many approved drugs have been reported to cause SSR, which has prompted withdrawal of drugs from the market. Common drugs that have been associated with SSR include nonsteroidal anti-inflammatory drugs (NSAIDs), sulfonamides, anticonvulsants, allopurinol, and antimalarials.

There is a need for markers that can predict the existence of or predisposition to SSR. Several studies have identified genetic risk factors for drug-related severe adverse events. However, there is currently no clinically useful method for predicting what drugs will cause SSR and in which patients.

### SUMMARY OF THE INVENTION

An aspect of the invention provides a method for predicting the risk of a patient for developing adverse drug reactions, particularly Serious Skin Rash (SSR), which includes severe adverse reactions such as Stevens-Johnson Syndrome (SJS) and Toxic Epidermal Necrolysis (TEN).

SSR may be caused by drugs such as nonsteroidal anti-inflammatory agents (NSAIDs), sulfonamides, anticonvulsants, allopurinol, and antimalarials.

Another aspect of the invention provides a method of identifying a subject afflicted with or at risk of developing SSR comprising (a) obtaining a nucleic acid-containing sample from the subject; and (b) analyzing at least one genetic marker, wherein the presence of the at least one genetic marker indicates that the subject is afflicted with or at risk of developing SSR. The method may further comprise treating the subject based on the results of step (b). The method may further comprise taking a clinical history from the subject. Genetic markers that are useful for the invention include, but are not limited to, alleles, microsatellites, SNPs, and haplotypes. The sample may be any sample capable of being obtained from a subject, including but not limited to serum, sputum, saliva, mucosal scraping, tissue biopsy samples, lacrimal secretion, semen, and sweat.

In some embodiments of the invention, the genetic markers are SNPs selected from those listed in Tables 1, 2, 3, 4, 5 and 8. In other embodiments, genetic markers that are linked to each of the SNPs can be used to predict the corresponding SSR risk.

The presence of the genetic marker can be detected using any method known in the art. Analysis may comprise nucleic acid amplification, such as PCR. Analysis may also comprise primer extension, restriction digestion, sequencing, hybridization, a DNAse protection assay, mass spectrometry, labeling, and separation analysis.

### BRIEF DESCRIPTION OF THE FIGURES

FIG. 1 is a Manhattan plot that summarizes the genome-wide association result for the R1 data set. Each dot in the plot represents an SNP, the x-axis refers to its position on chromosomes (human NCBI build 36), and the y-axis refers to the −log 10 (p-value) of the SNP from the trend test in the case/control associate study.

FIG. 2 is a Manhattan plot that summarizes the genome-wide association result for the R1+POPRES+HapMap data set. Each dot in the plot represents an SNP, the x-axis refers to its position on chromosomes (human NCBI build 36), and the y-axis refers to the −log 10 (p-value) of the SNP from the trend test in the case/control associate study.

FIG. 3 is a Manhattan plot that summarizes the genome-wide association result for R1+POPRES+HapMap+iControlDB data set. Each dot in the plot represents an SNP, the x-axis refers to its position on chromosomes (human NCBI build 36), and the y-axis refers to the −log 10 (p-value) of the SNP from the trend test in the case/control associate study.

FIG. 4 is a plot showing the population structure of SSR cohorts for a genome-wide association study for the R1 (plus signs (+), 52 cases, 96 controls)+Italian cohort (x's, 19 cases)+HapMap TSI (circles, 88 controls)+POPRES (squares, 21 controls)+Lamotrigine cohort (diamonds, 5 cases, 52 controls) data set.

FIG. 5 is a Manhattan plot that summarizes the genome-wide association result for the n-EU SSR data set. Each dot in the plot represents an SNP, the x-axis refers to its position on chromosomes (human NCBI build 36), and the y-axis refers to the −log 10 (p-value) of the SNP from the trend test in the case/control associate study.

FIG. 6 is a qq-plot of the chi-square statistics from the genome-wide association studies for the n-EU SSR data set. The solid straight line denotes the null model, and the dashed lines mark the 95% confidence intervals of the null model. Each dot in the plot represents an SNP, the x-axis refers to the expected chi-square values from the null model and the y-axis refers to the observed chi-square values. Dots outside dashed lines represent significant deviations from the null model.

FIG. 7 is a Manhattan plot that summarizes the genome-wide association result for the s-EU SSR data set. Each dot in the plot represents an SNP, the x-axis refers to its position on chromosomes (human NCBI build 36), and the y-axis refers to the −log 10 (p-value) of the SNP from the trend test in the case/control associate study.

FIG. 8 is a qq-plot of the chi-square statistics from the genome-wide association studies for the s-EU SSR data set. The solid straight line denotes the null model, and the dashed lines mark the 95% confidence intervals of the null model. Each dot in the plot represents an SNP, the x-axis refers to the expected chi-square values from the null model and the y-axis refers to the observed chi-square values. Dots outside dashed lines represent significant deviations from the null model.

FIG. 9(a) is a plot showing the population structure of all subjects from three collections. The circles represent Caucasian subjects, the squares represent subjects of other ethnicities. FIG. 9(b) is a plot showing the population structure of Caucasian subjects. The first two eigen vectors separate the Europeans into UK cluster (top), Italian cluster (lower center) and Eastern Europeans (lower right). The cluster on the lower left are POPRES of Spanish origin.

FIG. 10(a) is a Manhattan plot that summarizes the genome-wide association result from overall European cases and controls. Each dot in the plot represents an SNP, the x-axis refers to its position on chromosomes (human NCBI build 36), and the y-axis refers to the −log 10 (p-value) of the SNP from the logistic regression test. FIG. 10(b) is a quantile-quantile plot of −log₁₀ of p-values against the expected values under the null model. The bulk of the values (thick line) closely follows the expectation under the null model (thin line).

FIG. 11 is a plot showing improved power by expanding the control set. The power was defined as the proportion of simulations where p-values were smaller than the two cutoffs (1×10⁻⁶ and 5×10⁻⁸) with 49 cases and the number of controls in the x axis, assuming the odds ratio of the associated SNP was 3.5 and the minor allele frequency was 0.1 (conditions similar to the top associated SNP from the n-EU group). The top line of dots represents the power using p-value cutoff of 1×10⁻⁶, and the bottom line of dots represents the power using p-value cutoff of 5×10⁻⁸.

### DETAILED DESCRIPTION OF THE INVENTION

For the purposes of promoting an understanding of the principles of the invention, reference will now be made to specific embodiments and specific language will be used to describe the same. It will nevertheless be understood that no limitation of the scope of the invention is thereby intended, and that such alterations and further modifications of the invention, and such further applications of the principles of the invention as illustrated herein as would normally occur to one skilled in the art to which the invention relates, are contemplated as within the scope of the invention.

All terms as used herein are defined according to the ordinary meanings they have acquired in the art. Such definitions can be found in any technical dictionary or reference known to the skilled artisan, such as the McGraw-Hill Dictionary of Scientific and Technical Terms (McGraw-Hill, Inc.), Molecular Cloning: A Laboratory Manual (Cold Springs Harbor, N.Y.), Remington's Pharmaceutical Sciences (Mack Publishing, PA), and Stedman's Medical Dictionary (Williams and Wilkins, MD). These references, along with those references, patents, and patent applications cited herein are hereby incorporated by reference in their entirety.

The term “marker” as used herein refers to any morphological, biochemical, or nucleic acid-based phenotypic difference which reveals a DNA polymorphism. The presence of markers in a sample may be useful to determine the phenotypic status of a subject (e.g., whether an individual has or has not been afflicted with SSR), or may be predictive of a physiological outcome (e.g., whether an individual is likely to develop SSR). The markers may be differentially present in a biological sample or fluid, such as blood plasma or serum. The markers may be isolated by any method known in the art, including methods based on mass, binding characteristics, or other physicochemical characteristics. As used herein, the term “detecting” includes determining the presence, the absence, or a combination thereof, of one or more markers.

Non-limiting examples of nucleic acid-based, genetic markers include alleles, microsatellites, single nucleotide polymorphisms (SNPs), haplotypes, copy number variants (CNVs), insertions, and deletions.

The term “allele” as used herein refers to an observed class of DNA polymorphism at a genetic marker locus. Alleles may be classified based on different types of polymorphism, for example, DNA fragment size or DNA sequence. Individuals with the same observed fragment size or same sequence at a marker locus have the same genetic marker allele and thus are of the same allelic class.

The term “locus” as used herein refers to a genetically defined location for a collection of one or more DNA polymorphisms revealed by a morphological, biochemical or nucleic acid-bred analysis.

The term “genotype” as used herein refers to the allelic composition of an individual at genetic marker loci under study, and “genotyping” refers to the process of determining the genetic composition of individuals using genetic markers.

The term “single nucleotide polymorphism” (SNP) as used herein refers to a DNA sequence variation occurring when a single nucleotide in the genome or other shared sequence differs between members of a species or between paired chromosomes in an individual. The difference in the single nucleotide is referred to as an allele. A “haplotype” as used herein refers to a set of single SNPs on a single chromatid that are statistically associated.

The term “microsatellite” as used herein refers to polymorphic loci present in DNA that comprise repeating units of 1-6 base pairs in length.

An aspect of the invention provides a method for predicting the risk of a patient for developing adverse drug reactions, particularly SSR. As used herein, an “adverse drug reaction” is as an undesired and unintended effect of a drug. A “drug” as used herein is any compound or agent that is administered to a patient for prophylactic, diagnostic or therapeutic purposes.

SSR may be caused by many different classes of drugs. Nonlimiting examples of drugs known to cause SSR include nonsteroidal anti-inflammatory agents (NSAIDs), sulfonamides, anticonvulsants, allopurinol, and antimalarials.

Another aspect of the invention provides a method of identifying a subject afflicted with or at risk of developing SSR comprising (a) obtaining a nucleic acid-containing sample from the subject; and (b) analyzing at least one genetic marker, wherein the presence of the at least one genetic marker indicates that the subject is afflicted with, or at risk of developing, SSR. The method may further comprise treating the subject based on the results of step (b). The method may further comprise taking a clinical history from the subject. Genetic markers that are useful for the invention include, but are not limited to, alleles, microsatellites, SNPs, haplotypes, CNVs, insertions, and deletions.

In some embodiments of the invention, the genetic markers are one or more SNPs selected from those listed in Tables 1, 2, 3, 4, 5 and 8. The reference numbers provided for these SNPs are from the NCBI SNP database, at www.ncbi.nlm.nih.gov/entrez/query.fcgi?db=snp.

Each person's genetic material contains a unique SNP pattern that is made up of many different genetic variations. SNPs may serve as biological markers for pinpointing a disease on the human genome map, because they are usually located near a gene found to be associated with a certain disease. Occasionally, a SNP may actually cause a disease and, therefore, can be used to search for and isolate the disease-causing gene.

In accordance with the invention, at least one marker may be detected. It is to be understood, and is described herein, that one or more markers may be detected and subsequently analyzed, including several or all of the markers identified. Further, it is to be understood that the failure to detect one or more of the markers of the invention, or the detection thereof at levels or quantities that may correlate with SSR, may be useful and desirable as a means of selecting the individuals afflicted with or at risk for developing SSR, and that the same forms a contemplated aspect of the invention.

In addition to the SNPs listed in Tables 1, 2, 3, 4, 5 and 8, genetic markers that are linked to each of the SNPs may be used to predict the corresponding SSR risk as well. The presence of equivalent genetic markers may be indicative of the presence of the allele or SNP of interest, which, in turn, is indicative of a risk for SSR. For example, equivalent markers may co-segregate or show linkage disequilibrium with the marker of interest. Equivalent markers may also be alleles or haplotypes based on combinations of SNPs.

The equivalent genetic marker may be any marker, including alleles, microsatellites, SNPs, and haplotypes. In some embodiments, the useful genetic markers are about 200 kb or less from the locus of interest. In other embodiments, the markers are about 100 kb, 80 kb, 60 kb, 40 kb, or 20 kb or less from the locus of interest.

To further increase the accuracy of risk prediction, the marker of interest and/or its equivalent marker may be determined along with the markers of accessory molecules and co-stimulatory molecules which are involved in the interaction between antigen-presenting cell and T-cell interaction. For example, the accessory and co-stimulatory molecules include cell surface molecules (e.g., CD80, CD86, CD28, CD4, CD8, T cell receptor (TCR), ICAM-1, CD11a, CD58, CD2, etc.), and inflammatory or pro-inflammatory cytokines, chemokines (e.g., TNF-α), and mediators (e.g., complements, apoptosis proteins, enzymes, extracellular matrix components, etc.). Also of interest are genetic markers of drug metabolizing enzymes which are involved in the bioactivation and detoxification of drugs. Non-limiting examples of drug metabolizing enzymes include phase I enzymes (e.g., cytochrome P450 superfamily), and phase II enzymes (e.g., microsomal epoxide hydrolase, arylamine N-acetyltransferase, UDP-glucuronosyl-transferase, etc.).

Another aspect of the invention provides a method for pharmacogenomic profiling. Accordingly, a panel of genetic factors is determined for a given individual, and each genetic factor is associated with the predisposition for a disease or medical condition, including adverse drug reactions. In some embodiments, the panel of genetic factors may include at least one SNP selected from Tables 1, 2, 3, 4, 5 and 8. The panel may include equivalent markers to the markers in Tables 1, 2, 3, 4, 5 and 8. The genetic markers for accessory molecules, co-stimulatory molecules and/or drug metabolizing enzymes described above may also be included.

Yet another aspect of the invention provides a method of screening and/or identifying agents that can be used to treat SSR by using any of the genetic markers of the invention as a target in drug development. For example, cells expressing any of the SNPs or equivalents thereof may be contacted with putative drug agents, and the agents that bind to the SNP or equivalent are likely to inhibit the expression and/or function of the SNP. The efficacy of the candidate drug agent in treating SSR may then be further tested.

In some embodiments, it may be desirable to amplify the target sequence before evaluating the genetic marker. Nucleic acids used as a template for amplification may be isolated from cells, tissues or other samples according to standard methodologies such as are described, for example, in Sambrook et al., 1989. In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” refers to any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form.

For amplification of SNPs, pairs of primers designed to selectively hybridize to nucleic acids flanking the polymorphic site may be contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex may be contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

Any known template dependent process may be advantageously employed to amplify the oligonucleotide sequences present in a given template sample. One of the best known amplification methods is the polymerase chain reaction (PCR), which is described in U.S. Pat. Nos. 4,683,195, 4,683,202 and 4,800,159, and in Innis et al., 1988, each of which is incorporated herein by reference in their entirety.

A reverse transcriptase PCR amplification procedure may be performed when the source of nucleic acid is fractionated or whole cell RNA. Methods of reverse transcribing RNA into cDNA are well known and are described in, for example, Sambrook et al., 1989. Alternative exemplary methods for reverse polymerization utilize thermostable DNA polymerases. These methods are described, for example, in International Publication WO 90/07641. Polymerase chain reaction methodologies are well known in the art. Representative methods of RT-PCR are described, for example, in U.S. Pat. No. 5,882,864.

Another method for amplification is ligase chain reaction (LCR), disclosed, for example, in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed, for example, in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (e.g., fewer than 1000 bases). A benefit to using this approach is that each individual region would not have to be amplified separately. There would be the potential to screen thousands of SNPs from the single PCR reaction.

Qbeta Replicase, described, for example, in International Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed, for example, in U.S. Pat. No. 5,916,779, is another method of carrying out isothermal amplification of nucleic acids which involves multiple rounds of strand displacement and synthesis, e.g., nick translation.

Other nucleic acid amplification procedures include polymerization-based amplification systems (TAS), for example, nucleic acid sequence based amplification (NASBA) and 3SR (Kwoh et al., 1989; International Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

International Application WO 89/06700 discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

### Methods of Detection

The genetic markers of the invention may be detected using any method known in the art. For example, genomic DNA may be hybridized to a probe that is specific for the allele of interest. The probe may be labeled for direct detection, or contacted by a second, detectable molecule that specifically binds to the probe. Alternatively, cDNA, RNA, or the protein product of the allele may be detected. For example, serotyping or microcytotoxity methods may be used to determine the protein product of the allele. Similarly, equivalent genetic markers may be detected by any methods known in the art.

It is within the purview of one of skill in the art to design genetic tests to screen for SSR or a predisposition for SSR based on analysis of the genetic markers of the invention. For example, a genetic test may be based on the analysis of DNA for SNP patterns. Samples may be collected from a group of individuals affected by SSR due to drug treatment and the DNA analyzed for SNP patterns. Non-limiting examples of sample sources include blood, sputum, saliva, mucosal scraping or tissue biopsy samples. These SNP patterns may then be compared to patterns obtained by analyzing the DNA from a group of individuals unaffected by SSR due to drug treatment. This type of comparison, called an “association study,” can detect differences between the SNP patterns of the two groups, thereby indicating which pattern is most likely associated with SSR. Eventually, SNP profiles that are characteristic of a variety of diseases will be established. These profiles can then be applied to the population at general, or those deemed to be at particular risk of developing SSR.

Various techniques may be used to assess genetic markers. Non-limiting examples of a few of these techniques are discussed here and also described in US Patent Publication 2007/026827, the disclosure of which is herein incorporated by reference in its entirety. In accordance with the invention, any of these methods may be used to design genetic tests for affliction with or predisposition to SSR. Additionally, these methods are continually being improved and new methods are being developed. It is contemplated that one of skill in the art will be able to use any improved or new methods, in addition to any existing method, for detecting and analyzing the genetic markers of the invention.

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even individual species members) from one another.

Restriction endonucleases are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in genetic marker analysis, such as SNP analysis, requires that the SNP affect cleavage of at least one restriction enzyme site.

Primer Extension is a technique in which the primer and no more than three NTPs may be combined with a polymerase and the target sequence, which serves as a template for amplification. By using fewer than all four NTPs, it is possible to omit one or more of the polymorphic nucleotides needed for incorporation at the polymorphic site. The amplification may be designed such that the omitted nucleotide(s) is(are) not required between the 3′ end of the primer and the target polymorphism. The primer is then extended by a nucleic acid polymerase, such as Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

Oligonucleotide Hybridization is a technique in which oligonucleotides may be designed to hybridize directly to a target site of interest. The hybridization can be performed on any useful format. For example, oligonucleotides may be arrayed on a chip or plate in a microarray. Microarrays comprise a plurality of oligos spatially distributed over, and stably associated with, the surface of a substantially planar substrate, e.g., a biochip. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., a target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used is known in the art. The probe molecules of the arrays which are capable of sequence-specific hybridization with target nucleic acid may be polynucleotides or hybridizing analogues or mimetics thereof, including: nucleic acids in which the phosphodiester linkage has been replaced with a substitute linkage, such as phosphorothioate, methylimino, methylphosphonate, phosphoramidate, guanidine and the like; and nucleic acids in which the ribose subunit has been substituted, e.g., hexose phosphodiester, peptide nucleic acids, and the like. The length of the probes will generally range from 10 to 1000 nts, wherein in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

Probe molecules arrayed on the surface of a substrate may correspond to selected genes being analyzed and be positioned on the array at a known location so that positive hybridization events may be correlated to expression of a particular gene in the physiological source from which the target nucleic acid sample is derived. The substrate with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. Different array configurations and methods for their production and use are known to those of skill in the art and disclosed, for example, in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755, the disclosures of which are herein incorporated by reference in their entireties.

Following hybridization, where non-hybridized labeled nucleic acid is capable of emitting a signal during the detection step, a washing step is employed in which unhybridized labeled nucleic acid is removed from the support surface, generating a pattern of hybridized nucleic acid on the substrate surface. Various wash solutions and protocols for their use are known to those of skill in the art and may be used.

Where the label on the target nucleic acid is not directly detectable, the array comprising bound target may be contacted with the other member(s) of the signal producing system that is being employed. For example, where the target is biotinylated, the array may be contacted with streptavidin-fluorescer conjugate under conditions sufficient for binding between the specific binding member pairs to occur. Following contact, any unbound members of the signal producing system will then be removed, e.g., by washing. The specific wash conditions employed will depend on the specific nature of the signal producing system that is employed, as will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, the potential for a mismatch hybridization event that could potentially generate a false positive signal on the pattern may be reduced by treating the array of hybridized target/probe complexes with an endonuclease under conditions sufficient such that the endonuclease degrades single stranded, but not double stranded, DNA. Various different endonucleases are known and may be used, including but not limited to mung bean nuclease, Si nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described herein, the resultant hybridization pattern may be detected. In detecting or visualizing the hybridization pattern, the intensity or signal value of the label may also be quantified, such that the signal from each spot of the hybridization will be measured and compared to a unit value corresponding the signal emitted by known number of labeled target nucleic acids to obtain a count or absolute value of the copy number of each end-labeled target that is hybridized to a particular spot on the array in the hybridization pattern.

It will be appreciated that any useful system for detecting nucleic acids may be used in accordance with the invention. For example, mass spectrometry, hybridization, sequencing, labeling, and separation analysis may be used individually or in combination, and may also be used in combination with other known methods of detecting nucleic acids.

Electrospray ionization (ESI) is a type of mass spectrometry that is used to produce gaseous ions from highly polar, mostly nonvolatile biomolecules, including lipids. The sample is typically injected as a liquid at low flow rates (1-10 μL/min) through a capillary tube to which a strong electric field is applied. The field charges the liquid in the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice. A potential difference of between 1 to 5 kV (but more typically 2 to 3 kV) is applied to the capillary by power supply to generate a high electrostatic field (10⁶ to 10⁷ V/m) at the capillary tip. A sample liquid, carrying the analyte to be analyzed by the mass spectrometer, is delivered to the tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multi-charged gas phase ions in the form of an ion beam. The ions are then collected by the grounded (or oppositely-charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals and bioactive peptides.

Secondary ion mass spectroscopy (SIMS) is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analyzed by the mass spectrometer in this method.

Laser desorption mass spectroscopy (LD-MS) involves the use of a pulsed laser, which induces desorption of sample material from a sample site, and effectively, vaporizes sample off of the sample substrate. This method is usually used in conjunction with a mass spectrometer, and can be performed simultaneously with ionization by adjusting the laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to their different sizes causing different velocities. Each ion mass will thus have a different flight-time to the detector.

Other advantages of the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive and robust, and has been shown to be capable of detecting trace levels in natural samples without any prior extraction preparations.

Matrix Assisted Laser Desorption/Ionization Time-of Flight (MALDI-TOF) is a type of mass spectrometry useful for analyzing molecules across an extensive mass range with high sensitivity, minimal sample preparation and rapid analysis times. MALDI-TOF also enables non-volatile and thermally labile molecules to be analyzed with relative ease. One important application of MALDI-TOF is in the area of quantification of peptides and proteins, such as in biological tissues and fluids.

Surface Enhanced Laser Desorption and Ionization (SELDI) is another type of desorption/ionization gas phase ion spectrometry in which an analyte is captured on the surface of a SELDI mass spectrometry probe. There are several known versions of SELDI.

One version of SELDI is affinity capture mass spectrometry, also called Surface-Enhanced Affinity Capture (SEAC). This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” The capture reagent may be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

Another version of SELDI is Surface-Enhanced Neat Desorption (SEND), which involves the use of probes comprising energy absorbing molecules that are chemically bound to the probe surface. Energy absorbing molecules (EAM) refer to molecules that are capable of absorbing energy from a laser desorption/ionization source and, thereafter, of contributing to desorption and ionization of analyte molecules in contact therewith. The EAM category includes molecules used in MALDI, frequently referred to as “matrix,” and is exemplified by cinnamic acid derivatives such as sinapinic acid (SPA), cyano-hydroxy-cinnamic acid (CHCA) and dihydroxybenzoic acid, ferulic acid, and hydroxyaceto-phenone derivatives. In certain versions, the energy absorbing molecule is incorporated into a linear or cross-linked polymer, e.g., a polymethacrylate. For example, the composition may be a co-polymer of α-cyano-4-methacryloyloxycinnamic acid and acrylate. In another version, the composition may be a co-polymer of α-cyano-4-methacryloyloxycinnamic acid, acrylate and 3-(tri-ethoxy)silyl propyl methacrylate. In another version, the composition may be a co-polymer of α-cyano-4-methacryloyloxycinnamic acid and octadecylmethacrylate (“C18 SEND”).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light. SEPAR and other forms of SELDI are readily adapted to detecting a marker or marker profile, in accordance with the present invention.

In accordance with the invention, nucleic acid hybridization is another useful method of analyzing genetic markers. Nucleic acid hybridization is generally understood as the ability of a nucleic acid to selectively form duplex molecules with complementary stretches of DNAs and/or RNAs. Depending on the application, varying conditions of hybridization may be used to achieve varying degrees of selectivity of the probe or primers for the target sequence.

Typically, a probe or primer of between 10 and 100 nucleotides, and up to 1-2 kilobases or more in length, will allow the formation of a duplex molecule that is both stable and selective. Molecules having complementary sequences over contiguous stretches greater than 20 bases in length may be used to increase stability and selectivity of the hybrid molecules obtained. Nucleic acid molecules for hybridization may be readily prepared, for example, by directly synthesizing the fragment by chemical means or by introducing selected sequences into recombinant vectors for recombinant production.

For applications requiring high selectivity, relatively high stringency conditions may be used to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated by those of skill depending on the desired results.

It is within the purview of the skilled artisan to design and select the appropriate primers, probes, and enzymes for any of the methods of genetic marker analysis. For example, for detection of SNPs, the skilled artisan will generally use agents that are capable of detecting single nucleotide changes in DNA. These agents may hybridize to target sequences that contain the change. Or, these agents may hybridize to target sequences that are adjacent to (e.g., upstream or 5′ to) the region of change.

In general, it is envisioned that the probes or primers described herein will be useful as reagents in solution hybridization for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest, as described herein, is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section are incorporated herein by reference.

The synthesis of oligonucleotides for use as primers and probes is well known to those of skill in the art. Chemical synthesis can be achieved, for example, by the diester method, the triester method, the polynucleotide phosphorylase method and by solid-phase chemistry. Various mechanisms of oligonucleotide synthesis have been disclosed, for example, in U.S. Pat. Nos. 4,659,774, 4,816,571, 5,141,813, 5,264,566, 4,959,463, 5,428,148, 5,554,744, 5,574,146, 5,602,244, each of which is incorporated herein by reference in its entirety.

In certain embodiments, nucleic acid products are separated by agarose, agarose-acrylamide or polyacrylamide gel electrophoresis using standard methods such as those described, for example, in Sambrook et al., 1989. Separated products may be cut out and eluted from the gel for further manipulation. Using low melting point agarose gels, the skilled artisan may remove the separated band by heating the gel, followed by extraction of the nucleic acid.

Separation of nucleic acids may also be effected by chromatographic techniques known in the art. There are many kinds of chromatography that may be used in the practice of the present invention, non-limiting examples of which include capillary adsorption, partition, ion-exchange, hydroxylapatite, molecular sieve, reverse-phase, column, paper, thin-layer, and gas chromatography, as well as HPLC.

A number of the above separation platforms may be coupled to achieve separations based on two different properties. For example, some of the primers may be coupled with a moiety that allows affinity capture, and some primers remain unmodified. Modifications may include a sugar (for binding to a lectin column), a hydrophobic group (for binding to a reverse-phase column), biotin (for binding to a streptavidin column), or an antigen (for binding to an antibody column). Samples may be run through an affinity chromatography column. The flow-through fraction is collected, and the bound fraction eluted (by chemical cleavage, salt elution, etc.). Each sample may then be further fractionated based on a property, such as mass, to identify individual components.

In certain aspects, it will be advantageous to employ nucleic acids of defined sequences of the present invention in combination with an appropriate means, such as a label, for determining hybridization. Various appropriate indicator means are known in the art, including fluorescent, radioactive, enzymatic or other ligands, such as avidin/biotin, which are capable of being detected. In the case of enzyme tags, colorimetric indicator substrates are known that may be employed to provide a detection means that is visibly or spectrophotometrically detectable, to identify specific hybridization with complementary nucleic acid containing samples. In yet other embodiments, the primer has a mass label that can be used to detect the molecule amplified. Other embodiments also contemplate the use of Taqman™ and Molecular Beacon™ probes.

Radioactive isotopes useful for the invention include, but are not limited to, tritium, ¹⁴C and ³²P. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

The choice of label may vary, depending on the method used for analysis. When using capillary electrophoresis, microfluidic electrophoresis, HPLC, or LC separations, either incorporated or intercalated fluorescent dyes may be used to label and detect the amplification products. Samples are detected dynamically, in that fluorescence is quantitated as a labeled species moves past the detector. If an electrophoretic method, HPLC, or LC is used for separation, products can be detected by absorption of UV light. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal may be monitored dynamically. Detection with a radioisotope or enzymatic reaction may require an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer, no label is required because nucleic acids are detected directly.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

While the foregoing specification teaches the principles of the invention, with examples provided for the purpose of illustration, it will be appreciated by one skilled in the art from reading this disclosure that various changes in form and detail can be made without departing from the true scope of the invention.

### EXAMPLES

### Example 1

### Whole-Genome Association (WGA) Study

A whole-genome association (WGA) study was undertaken in which the case group comprised 71 SSR cases that were exposed to a variety of drugs and 135 clinically matched controls, all contributed by GlaxoSmithKline (GSK). All cases and controls were genotyped using Illumina Human 1M chips. To prevent spurious association caused by population stratification, 56 Caucasian cases and 107 Caucasian controls were chosen based on Principal Component Analysis (PCA). 4 cases and 11 controls were removed from the study based on hidden relatedness and inconsistent gender. The genotypes of the remaining 52 cases and 96 controls were denoted as the R1 data set.

Illumina 1M chips include a total of 1,072,820 probes, including probes for Single-Nucleotide Polymorphisms (SNPs) and Copy Number Variations (CNVs). To ensure the quality of the analysis, all probes with Minor Allele Frequency (MAF) smaller than 0.01, missing call rate larger than 0.05, or p-value of Hardy-Weinberg Equilibrium (HWE) smaller than 10⁻⁷ were removed. The Cochran-Armitage trend test was applied on the remaining 866,880 SNPs. 4 SNPs were identified as having p-values of less than 10⁻⁵.

To improve the power of the association study, the R1 data set was combined with population controls, including 443 POPRES Caucasian subjects (POPRES is a set of control samples collected by GSK for general association studies), 105 HapMap III CEU subjects (subjects of northern European origin from phase III of the HapMap project, as described at http://www.hapmap.org/), and 2676 iControlDB Caucasian samples (iControlDB is a repository for genotyping control data generated by researchers using Illumina genotyping products). The POPRES and HapMap samples were genotyped with Illumina 1M chips and the iControlDB samples were genotyped with Illumina 550K chips. The probes on the 550K chips are a subset of the ones on the 1M chips. To maximize the utility of the population controls, two combined sets were prepared: R1+POPRES+HapMap, all using a 1M chip; and R1+all population controls. For each set, only the common SNPs that passed quality controls in each individual set were kept for GWAS.

The same type of quality control steps were applied on the combined sets. Because genotype data from different sources and experiments may contain different errors, two additional quality control steps were added. The first was to remove all SNPs that were A/T or G/C to avoid confusion about DNA strands. The second was to remove SNPs that appeared to have significantly different MAF between matched controls and population controls. Specifically, the control status was switched to case for the matched controls, trend tests were performed on the matched controls versus population controls, and any SNPs that had a p-value of 0.01 or less were removed. In the end result, 833,982 SNPs were kept in the R1+POPRES+HapMap set, and 627,140 SNPs were kept in the R1+POPRES+HapMap+iControlDB set.

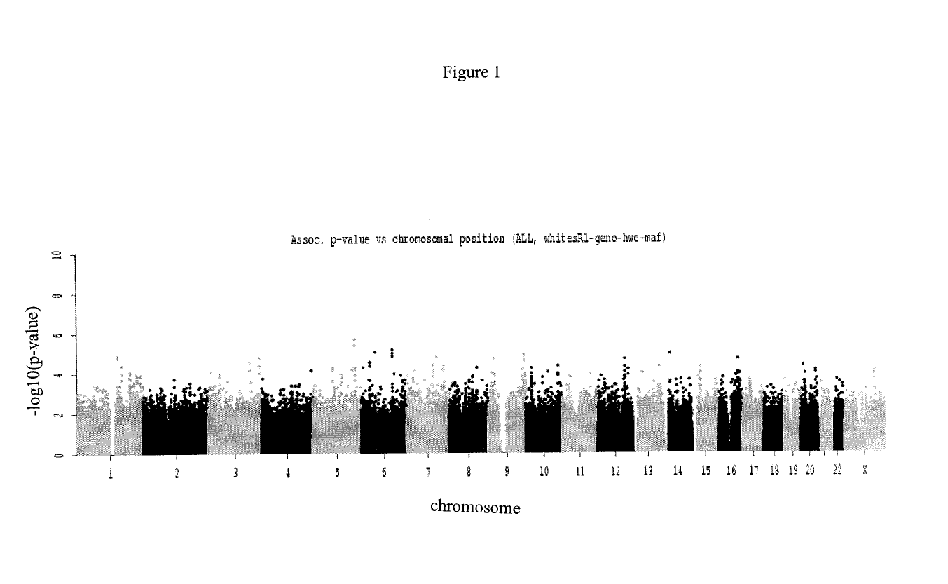

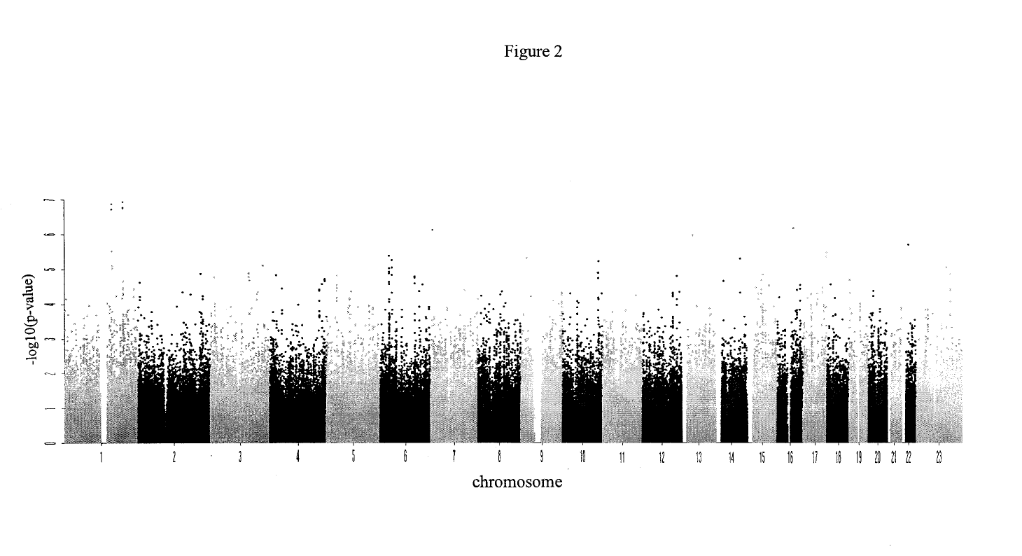

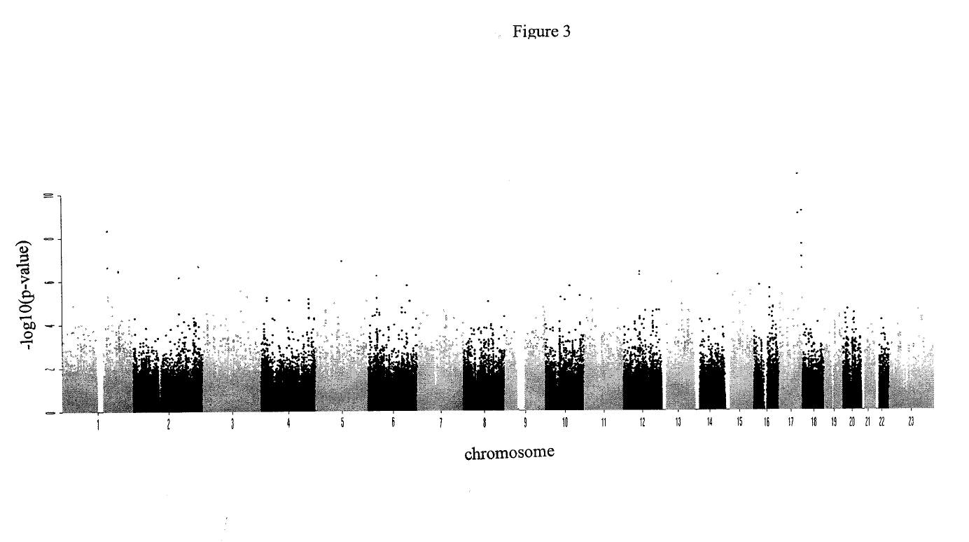

Tables 1, 2, and 3 show the SNPs that have a p-value smaller than 10⁻⁵ in each of the three data sets: R1, R1+POPRES+HapMap, and R1+POPRES+HapMap+iControlDB. FIGS. 1-3 are Manhattan plots summarizing the results of these studies. Table 4 shows the SNPS found to be the most strongly associated with SSR.

SNP rs4532807 has a p-value of 5×10⁻⁹, which is genome-wide statistically significant. The SNP is from chr 1 and is close to ATF6. ATF6 is an endoplasmic reticulum (ER) stress-regulated transmembrane transcription factor that activates the transcription of ER molecules. It is in the intron of ATF6.

SNP rs1984722 has a p-value of 1.4×10⁻¹¹ and odds ratio of 6.7, which is genome-wide statistically significant. The SNP is from chr 17 (pos: 64500261) and is within ABCA9, which is expressed during monocyte differentiation into macrophages. At least one paper (Shenton JM 2007) reported macrophages might be involved in early stages of immune response to skin rash.

SNP rs9898788 has a p-value of 7×10⁻¹⁹ and odds ratio of 4.8, which is genome-wide statistically significant. The SNP is from chr 17 (pos: 77849717) and is close to gene CD7, which plays an essential role in T-cell interactions and also in T-cell/B-cell interaction during early lymphoid development, and SECTM1, which might be involved in hematopoietic and/or immune system processes, and was expressed as a 1.8-kb mRNA in many of the tissues tested, with the highest level of expression in peripheral blood leukocytes (Slentz-Kesler et al 1998). The SNP is in the 17q25 region, which was reported to be associated with psoriasis.

SNP rs12629207 has a p-value of 10⁻⁹ and odds ratio of 6, which is genome-wide statistically significant. The SNP is from chr 3 and is close to the gene IL20RB, which is a receptor of IL20. IL20 receptors are expressed in skin and are dramatically up-regulated in psoriatic skin, and IL20 is involved in epidermal function and psoriasis (Blumberg et al. 2001).

SNP rs7758412 has a p-value of 9×10⁻¹² and odds ratio of 8, which is genome-wide statistically significant. The SNP is from chr 6 and is within the gene MOCS1 (molybdenum cofactor synthesis 1), which encodes a protein involved in molybdenum cofactor biosynthesis. A molybdenum-containing cofactor is essential to the function of 3 enzymes: sulfite oxidase, xanthine dehydrogenase, and aldehyde oxidase (Johnson et al. 1980).

In another WGA study, the R1 data set was combined with two cohorts: a Lamotrigine (trade name Lamictal®, an anticonvulsant marketed by GSK) cohort consisting of 6 cases and 63 drug-matched controls), and an Italian SSR cohort exposed to multiple drugs, consisting of 19 cases. Both cohorts were genotyped with Illumina 1M-duo chips.

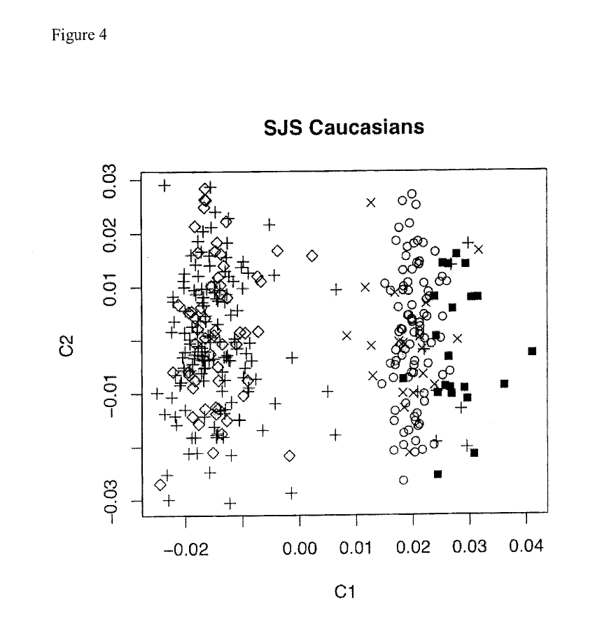

The same standard quality control procedures described before were applied to this WGA study. To delineate the population structure, the R1 data set was merged with the two cohorts, 88 HapMap TSI (representing central Italian population controls) genotypes, and 21 POPRES controls. Identical-By-State (IBS) and multidimensional scaling analysis (mds) via PLINK was performed and apparent non-Caucasian subjects were removed. The remaining Caucasian subjects were then re-analyzed, as shown in FIG. 4. In FIG. 4, the X-axis is the first dimension, representing the northern-southern separation of European population. Based on FIG. 4, the subjects were divided into two groups according to their position on the first dimension (cutoff at 0.005). The group on the left was labeled as the n-EU group, and the group on the right was labeled as the s-EU group. Statistical analyses were then conducted on each group separately. Table 5 shows the SNPs that have a p-value smaller than 10⁻⁵ in each of the following four data sets.

1. n-EU SSR Group

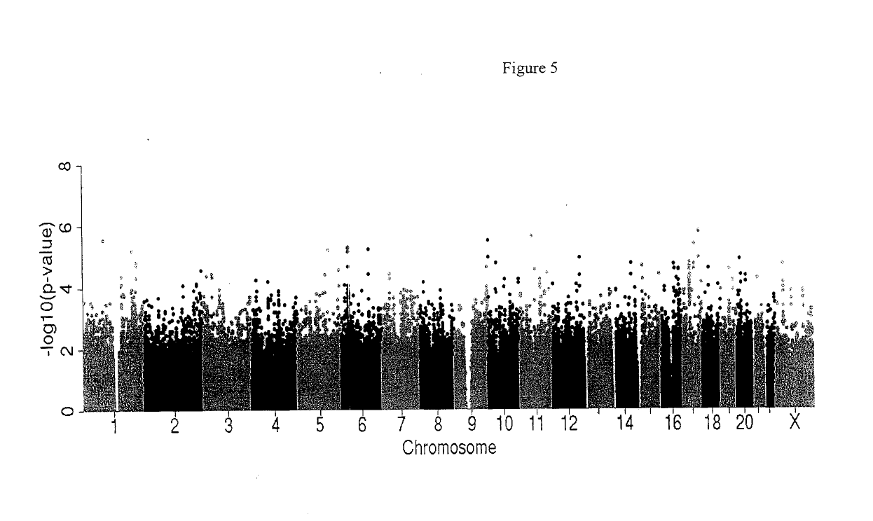

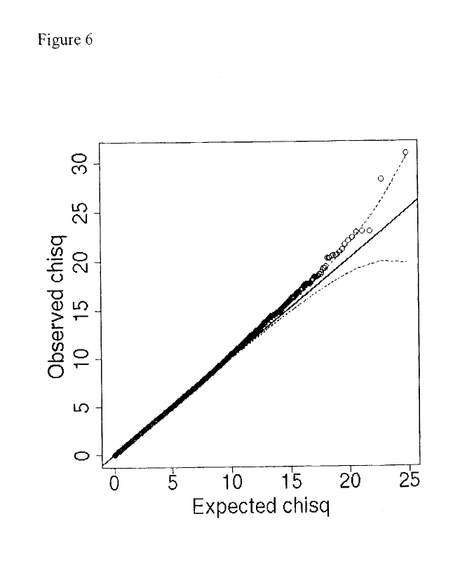

There are 52 cases and 143 controls in this group. The group was merged with another population control set, 200 UK subjects from the WTCCC2 (Wellcome Trust Case Control Consortium). IBS mds analysis indicated the merged set was still homogenous and there were no apparent population stratification issues. A Cochran-Armitage trend test was performed on the merged set (52 cases vs 343 controls). FIG. 5 is a Manhattan plot summarizing results from the test, and FIG. 6 is a qq-plot of the chi-square statistics from the test. A genome-wide significant SNP from chromosome 7, close to gene RPA3, was found: rs17137412, p-value=3×10⁻⁸, allelic OR=3.7; Minor Allele Frequency (MAF) in controls=0.1.

2. s-EU Group

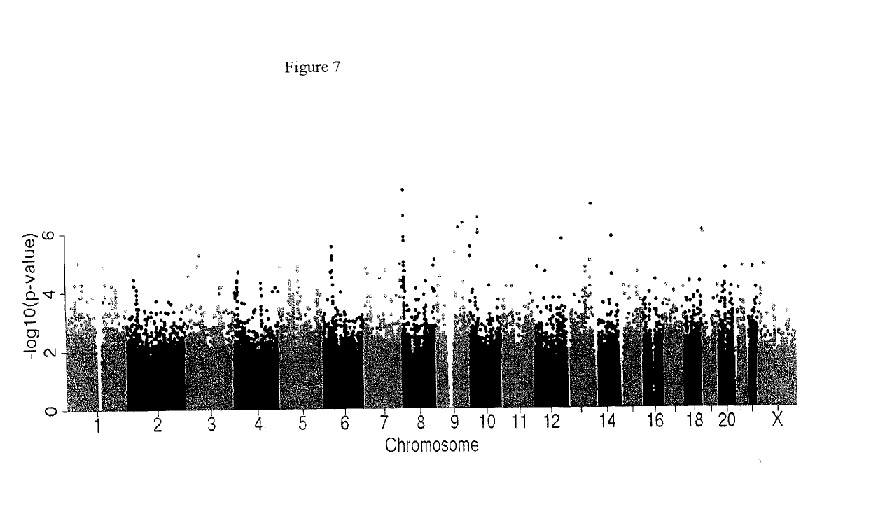

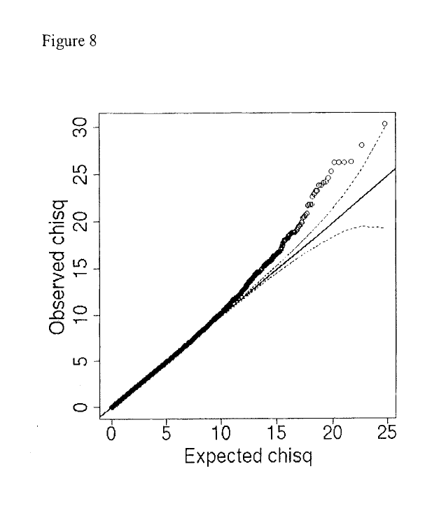

There are 21 cases and 115 controls in this group. The majority of the control subjects are from HapMap TSI, which were not genotyped together with the SSR cases. There are systematic differences in genotyping errors and insolvable DNA strand issues (for A/T or C/G SNPs) between subjects from such different sources. Because POPRES controls were genotyped in the same facility as SSR cases, 21 POPRES controls and 88 HapMap TSI controls were compared to identify the SNPs that are associated with different errors rather than by case/control status. Specifically, the POPRES controls were labeled as cases and the HapMap TSI subjects were labeled as controls in the trend test. A p-value <10⁻⁵ was set as the cutoff and problematic SNPs were selected. A Cochran-Armitage trend test was performed on the s-EU group and the problematic SNPs were removed. FIG. 7 is a Manhattan plot summarizing results from the test, and FIG. 8 is a qq-plot of the chi-square statistics from the test. A genome-wide significant SNP from chromosome 8, close to gene MSRA, was found: rs10098474, p-value=4×10⁻⁸, allelic OR=6, MAF in controls=0.17.

Additionally, two groups of cases separated by specific drugs were studied for genome-wide significant SNPs: Bactrim (14 cases) and Lamotrigine (9 cases).

### 1. Bactrim SSR Cases

All Bactrim SSR cases were included in the n-EU group described above. A trend test was performed on the 14 cases against the 343 controls in the n-EU group. Three genome-wide significant SNPs were found, as shown in Table 5: two are from chromosome 2, and the other is from chromosome 8.

### 2. Lamotrigine SSR Cases

All Lamotrigine cases were included in the n-EU group described above. A trend test was performed on the 9 cases versus the 143 controls. A genome-wide significant SNP from chromosome 7 was found: rs12019361, p-value=3×10⁻⁸, allelic OR=13, MAF in controls=0.06. The SNP is close to the gene ADAM22. Among 143 SSR controls (that is, excluding 200 WTCCC2 controls) in the n-EU group, there are 52 drug-matched controls. The MAF of this SNP in the 52 drug-matched controls is about 0.067.

### Example 2

### WGA Analysis

Using a similar data set as described in Example 1, a modified WGA analysis was performed as outlined below.

### Results

This study was based on three SJS/TEN collections: PGX40001 (Pirmohamed et al., 2007) and LAM30004 (Kazeema et al., 2009), and an Italian collection. Cases from PGX40001 and Italian collections were exposed to multiple drugs (Table 6). All subjects from LAM30004 were epilepsy patients treated with lamotrigine. The controls in PGX40001 were not matched by disease, but by age, gender, and ethnicity. The controls in LAM30004 were matched to cases by drug, age, and ethnicity. In total, 96 cases and 198 controls were genotyped at one facility in two batches: PGX40001 and LAM30004 subjects were in one batch and genotyped using the Illumina 1M chip, while the Italian collection was genotyped using the Illumina 1M-duo chip. Both chips contain more than 1 million probes of SNPs or CNVs. A series of quality control steps were applied to each collection separately to remove poor-quality probes based on minor allele frequency, rate of missing data, and deviations from Hardy-Weinberg expectations. Samples were subjected to a quality control procedure based on overall SNP call rate, gender and ethnicity consistency, and hidden relatedness from estimated identity-by-descent (IBD) (Purcell et al., 2007; Purcell, PLINK v1.04, http://pngu.mgh.harvard.edu/purcell/plink/). The Italian collection was genotyped without matching controls. Publicly available HapMap phase III TSI (subjects come from central Italy) were used as the population-matched controls for the Italian cases.

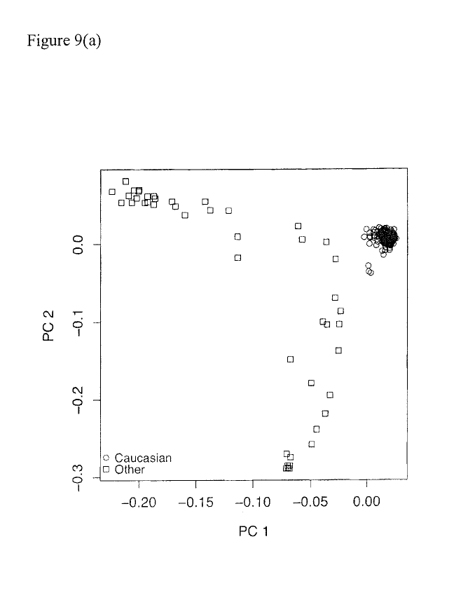

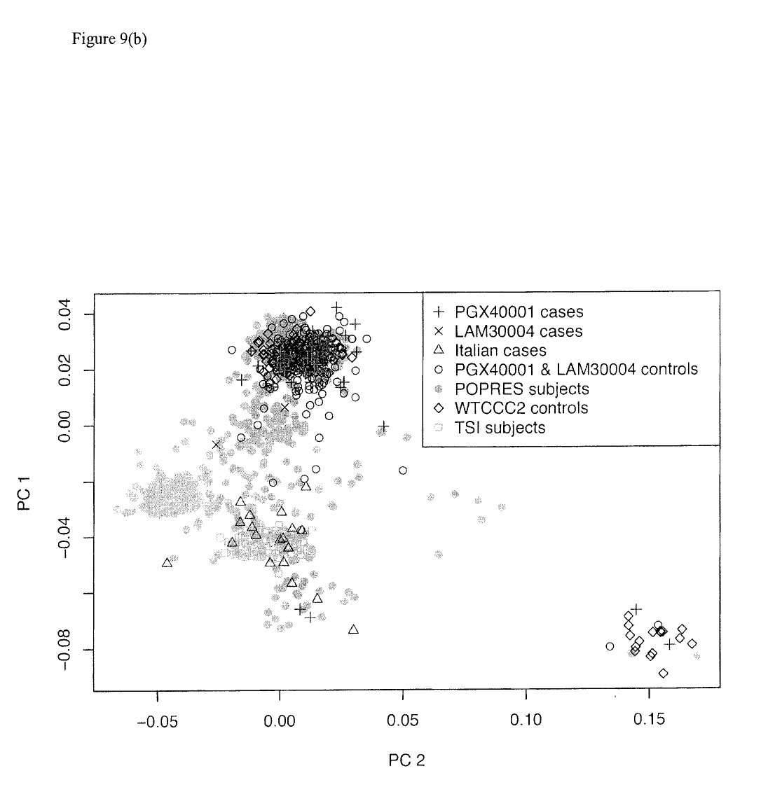

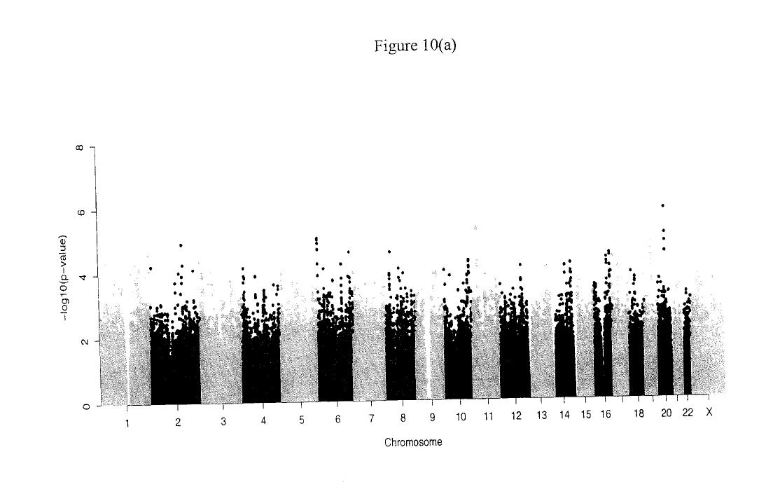

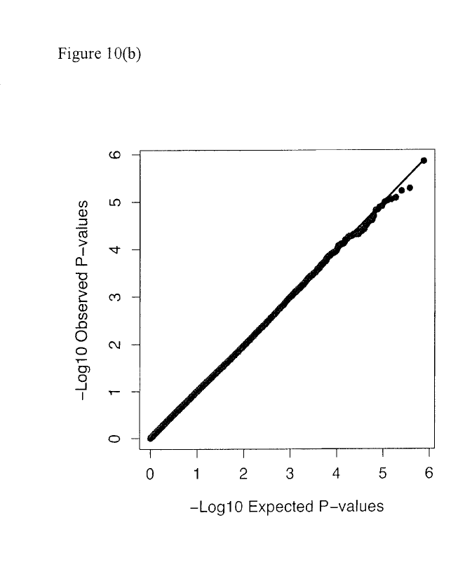

In total, 276 subjects passed quality controls, including 91 cases and 185 controls. Genetic structure in these subjects was investigated by principal components analysis (PCA) (FIG. 9a). Based on concordance of the PCA results and self-identified ancestry, selected 72 European cases, 162 controls and 88 HapMap TSI were selected as subjects for further analysis. To improve the power of the study and the ability to control for population stratification even within European subjects, the European cases and controls were combined with a set of 659 648 POPRES (Nelson et al., 2008) subjects, which represent sub-populations from the United Kingdom (UK), Spain, France Italy, and Eastern Europe. The POPRES subjects were genotyped in the same facility using Illumina 1M or 1M-Duo. Among the combined population, four PC axes were significant. The first two eigen vectors from PCA separate the subjects into UK cluster (on the top) and Italian cluster (on the lower left side) and East Europeans (on the lower right side) (FIG. 9b). For each case, up to 7 closest controls were selected based on eigen scores of the first four vectors. For the selected 72 cases and 461 controls, the association of single markers was then tested using logistic regression with first four eigen scores as covariates and sex under an additive model (FIG. 10). The accepted standards for significance amid genome-wide multiple testing was followed, setting a p-value cutoff at 5×10⁻⁸ to provide an approximate 5% significant level for each genome-wide analysis. The top associated SNP was rs6016348 on chromosome 20 (p-value=1.3×10⁻⁶; OR=2.9, 95% CI 1.9-4.6). Five other SNPs with a p-value smaller than 10⁻⁶ were found on chromosomes six and eleven, and reported in Table 8.

The subjects belonging to the UK cluster were then separated from those in the Italian cluster, and the association of the SNPs in each sub-group was tested.

### UK Group

The UK subjects from PGX40001 and LAM30004 collections only were analyzed first. There were 46 cases and 143 controls, including 59 males and 130 females. Associations were tested with 837,070 SNPs that passed quality control. No SNPs with p-values smaller than 10⁻⁶ were found. Interestingly, rs9501393, a missense polymorphism in the CYP21A2 gene within the major histocompatibility complex (MHC) region, had a p-value of 3.2×10⁻⁶ and OR of 4.0 (95% CI: 2.3-7.2).

### Italian Group

After combining the European cases and controls with a set of 648 POPRES (Nelson et al., 2008) and 88 controls were from HapMap TSI, 21 Italian cases, of whom 2 subjects belong to PGX40001, were identified. HapMap TSI subjects were not genotyped together with the SJS/TEN cases. To remove the SNPs that may have a systematic difference in genotyping errors, the allelic frequencies between the TSI and POPRES controls of Italian origin that had been genotyped in the same facility were compared. Specifically, we tested for significantly different allele frequency counts between the POPRES and TSI subjects using Fisher's exact test, and labeled all SNPs that have p-values smaller than 10⁻⁴ as problematic. 781,191 SNPs passed quality control. Applying a ratio of 6 between cases and controls, 97 control subjects, matched by the four significant eigen vectors, were included in this group. Associations were then tested by applying logistic regression with first four eigen scores and sex as covariates. No SNP with genome-wide significance or a p-value smaller than 10⁻⁶ was found.

### Improving Power by Expanding the Control Set Using External Population Controls

For this retrospective study, the number of cases was small and fixed. Due to the rarity of SJS/TEN, it is extremely difficult to identify and collect more cases to improve the power of the study in a timely fashion. Therefore, one way of improving the power of the association study is to expand the control set (Wellcome Trust Case Control Consortium 2007). There are multiple large publicly available data sets. The WTCCC2 data (phase 2, downloaded from http://www.ebi.ac.uk/ega) which were genotyped using the same platform as this study (Illumina 1M) was selected. Standard quality control procedures were applied on the WTCCC2 data, and the set was combined with the UK group, removing SNPs with ambiguous strands or having a significant difference from the original n-EU controls. In the combined set, there were 46 cases and 4251 controls. Two PCA were found significant among the extended UK population. Fisher's exact test was applied on the data set (FIG. 10). The top associated SNP is rs17137412 (chr 7, position 7767212, p-value=1.2×10⁻⁸, OR: 4.0, 95% CI of OR: 2.5-6.2; MAF in cases vs controls: 0.33 vs 0.11). This is an intronic SNP within the AC006465.3 hypothetical gene, and close to RPA3 and GLCCI1. Rs17137412 has only one high LD (r²>0.7) associated SNPs in Hap Map but it is not present in the combined dataset.

### Drug-Specific Groups

It may be possible to detect drug-specific risk alleles with small numbers of cases if the effect is large enough. To investigate this, two specific drugs in the UK group, bactrim (12 cases) and lamotrigine (9 cases), were investigated.

### Bactrim SJS/TEN Cases

In the 12 cases induced by bactrim, no SNPs with a p-value smaller than 10⁻⁶ were identified when compared to 143 controls (49 males and 106 females). Fisher Exact test was then applied on the combined dataset (12 cases versus 2251 controls). Increasing power with the number of controls did not permit observations of any significant SNP with p value less than 10⁻⁶. However, among the 34 top associated snps with p value less than 10⁻⁵, there were two intronic snps (rs241432, rs241430) within the TAP2 gene, with the MHC region.

### Lamotrigine SJS/TEN Cases

Comparison of the 9 cases due to lamotrigine with all 143 UK group controls (including 52 drug-matched controls) did not identify a SNP with a p-value smaller than 10⁻⁶. Interestingly, the top associated SNP in an additive model (rs12019361, p=7×10⁻⁶, OR=12, MAF in all controls: 0.06, MAF in 52 lamotrigine-exposed controls: 0.067; Table 8) was intronic in ADAM22, which is a gene involved in epilepsy, a primary indication for this drug (Fukata et al., 2006). Fisher Exact test was then applied on the combined dataset (9 cases versus 2251 controls). The combined analysis confirmed rs12019361 as the top associated SNP (p value=8.183e-06, MAF in controls=0.06, OR=11.49 (95% CI 4.518-29.24)). Although all the cases were included, including the two that belonged to the cEU cluster, the result is not affected by population stratification because the MAF of rs12019361 in Central European controls is equal to the UK controls (MAF in 105 supposed cEU controls=0.07, MAF in 2251 supposed nEU controls=0.06).

Association with Copy Number Variations (CNVs)

Among the 192 Northern European individuals who passed the SNP quality control checks, 134 individuals (37 cases and 97 controls) passed stringent quality-control criteria for CNV calling. A total of 3233 CNVs were predicted, including 1062 duplications and 2171 deletions. On average, 24 CNV calls were made for each individual. The numbers and average size of both deletions and duplications did not appear to be significantly different between cases and controls. After multi-test correction, none of the common CNVs or those larger than 100 kb (neither deletions nor duplications) showed a significant association with the studied phenotype. There were 114 singleton CNVs (100 kb or larger; 49 deletions and 65 duplications), of which 29 were in cases. The singleton CNV rate did not appear to differ between cases and controls, but the average size of deletions were larger in cases than in controls (458.1 kb vs 202.7 kb, permuted p value=0.03). Eleven unique oversize (greater than 500 kb) CNVs were found, with three of them being greater than 1 MB (all cases p<0.02). Table 9 summarizes the unique oversize deletions and duplications.

### Power Calculations

Post hoc power calculations were conducted to better understand the power needed to detect associations and the potential benefit from expanding control sets in the context of our study.

A certain odds ratio (OR) value was assumed under an additive genetic model, and a range of minor allele frequencies in the population (MAF_{Popu}). The MAF in cases (MAF_{C}) was determined by the OR and MAF_{Popu}. The number of cases and controls were chosen to reflect the scenarios in the study (Table 10). All controls were assumed to be population controls, i.e., not matched by drug exposure and therefore containing subjects who could be cases had they been given the drug. The prevalence of SJS/TEN was set at 0.001, which is likely to be larger than the true value in the European population, therefore making the power estimation slightly conservative. The number of latent cases in the population controls was calculated as the product of the prevalence and the total number of controls. The numbers of minor alleles in cases and controls were determined by sampling independently from binomial distributions, with p at MAF_{C} and MAF_{Popu} respectively. For each combination of the condition, the sampling procedure was repeated 1000 times, and each time calculated the p-values using Fisher's exact test assuming additive models. The power was estimated as the fraction when the p-value is lower than cutoffs. Two cutoff values were chosen: 10⁻⁶ and 5×10⁻⁸ (genome-wide significance).

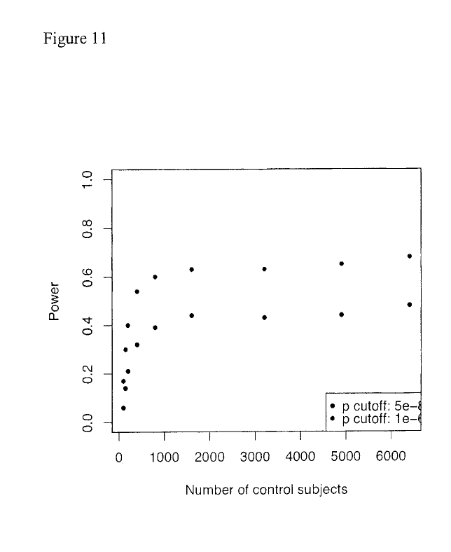

As expected, the power always increases with the increase in the number of controls. With 49 cases, assuming an OR of 3.5 and MAF_{Popu} of 0.1, which are the conditions that are similar to the top associated SNP in UK SJS/TEN group, the increase in power for detecting genome-wide significant (p-value <5×10⁻⁸) markers reaches a plateau of 0.33 at around 1600 controls. The power is 0.084 from the original sample size of 143 controls. Using publicly available external population controls increases the power by four fold (FIG. 11). Ninety cases are required to achieve a comparable increase in power given the original 143 controls. With a less stringent p-value cutoff at 10⁻⁶ which is useful for initial discovery of risk alleles, the power increases from 0.2 to 0.53, a 2.6-fold increase.

### Materials and Methods

### Subjects

### PGX40001 Collection

Cases were defined by Pirmohamed et al., 2007. Briefly, all cases were retrospectively enrolled and screened by expert review based on SJS/TEN inclusion and exclusion criteria. The controls were collected at the site of case matching for age, gender and ethnicity. In total, 71 cases and 135 controls were genotyped using Illumina 1M in the study.

### LAM30004 Collection:

Cases were defined by Kazeem et al., 2009. All cases and controls were epilepsy patients treated with lamotrigine and recruited retrospectively. Cases were patients who developed SJS/TEN or hypersensitivity reactions (although only SJS/TEN cases are included in this study), while controls were patients exposed to lamotrigine without developing SJS/TEN. In addition to matching for the drug of interest, the controls were also chosen based on other factors such as age, ethnicity, and concurrent valproic acid usage. SJS/TEN was defined by dermatologists using standard phenotypic criteria. The majority of subjects were Caucasians of Northern European origin. In total, 6 cases and 63 controls were included in this study.

### Italian Cases

Between November 2007 and March 2008, 19 retrospective cases of SJS due to a number of drugs were collected from Dermatology Department at the University of Florence and Dermatology Department at University of Verona. All cases were of self-reported Caucasian ethnic origin. Cases were identified by searching patient databases at these sites. A dermatologist's diagnosis was recorded in the database at the time of the reaction onset or at the discharge from the hospital. Patients with a diagnosis of either SJS or TEN were contacted. Once patients had signed a consent form, their eligibility was assessed by reference to their case-notes. Cases were defined based on three major clinical criteria: pattern of skin lesions, distribution of lesions, and percentage of epidermal detachment during the course of the disease. A diagnosis of SJS was considered if blistering did not exceed 10%, while TEN cases had greater than 30% of the body surface area blistered, with SJS-TEN overlap cases occupying an intermediate position (Bastuji-Garin et al., 2003). Exclusion criteria were (a) concomitant HIV infection, and (b) concomitant immunosuppressant drugs. Ethical approval was provided by the University of Florence Ethics committee.

### Control Selection

The controls from PGX40001 were collected at the same site as the cases, matched on the basis of ethnicity and gender. The controls in LAM30004 were matched on drug treatment as well as age and ethnicity. No specific control matching was done for the Italian cases. However, 88 HapMap phase III TSI subjects and 648 POPRES controls genotyped by the International Serious Adverse Events Consortium were as the controls for the Italian cases. About 4900 WTCCC2 subjects were analyzed and 4837 of these were chosen to match the Northern European cases.

### Genotyping

All subjects from PGX40001 and LAM30004 and POPRES controls were genotyped in one facility using the Illumina 1M chip. The Italian cases were genotyped using Illumina 1M-duo chip, which contains all markers on Illumina 1M chip. The chips contain about 1.07 and 1.2 million markers of SNPs and CNV probes, respectively. All genotyping was conducted using established protocols by Expression Analysis (Research Triangle Park, NC, USA). The genotypes of HapMap TSI were downloaded from http://hapmap.org public release #27. The genotypes of WTCCC2 subjects were downloaded from The European Genome-phenome Archive (http://www.ebi.ac.uk/ega/)—1958 British Birth Cohort and UK Blood Service Group (only Illumina 1M data).

### Genotype Quality Control

For each set of genotype data, a series of quality control steps were applied. Specifically, any marker that did NOT pass any of the following criteria was discarded:

1. Call rate greater than 95%

2. Minor allele frequency greater than 1%

3. A p-value for Hardy-Weinberg equilibrium greater than 10⁻⁷ in controls (if applicable)

After applying these criteria, 837,175 SNPs were left in the PGX40001 and LAM30004 subjects. The Italian collection was merged with HapMap TSI subjects. There were 781,191 SNPs left after removing those SNPs that had a significant allele frequency difference between HapMap TSI and POPRES Italians. All subjects had less than 10% of missing genotyping calls.

In addition, subjects that were highly related based on estimated identity-by-state (IBS) using PLINK v1.05 were identified. There were twenty-one samples with IBS sharing values larger than 0.2 and smaller than 0.9 with at least one other sample. Removing 12 samples resolved the issue (each of removed samples had lower overall SNP call rate than its related sample). There were four pairs of samples with almost identical genotypes (IBD sharing >0.99). These near-identical samples were further investigated by comparing the genotypes from this study with those from a previous experiment that included Affymetrix 500K SNP genotyping. Three samples (two were cases) were found to have significant inconsistency and were excluded. The fourth pair was also identical according to the Affymetrix 500K result and regarded as a true sample duplicate. The sample with the higher SNP call rate was retained. Additionally, two cases were removed due to inconsistency between reported ethnicity and that inferred by PCA (see next section). In total, 18 subjects were discarded, including 5 cases and 13 controls.

When combining data from external sources (such as WTCCC2 and HapMap), SNPs that had significantly different allele frequencies compared to the control set that was genotyped in the same facility as the cases (Fisher's exact test p-value <10⁻⁴) were removed. All SNPs with potential strand annotation issues were also discarded by this approach.

### Principal Components Analysis

The smartPCA program from the EIGENSTRAT package (version 3.0) was used to conduct PCA in order to expose population structure, select analysis subsets and choose genetically-matched controls. SNPs from four known regions (Novembre et al., 2008) of long-range linkage disequilibrium (LD) were removed before conducting PCA. The study genotype data was first combined with HapMap data to identify major ancestry groups (Europeans, Asians, and Africans). PCA was then conducted on Europeans (self-reported non-Hispanic white or European) only to separate Northern Europeans and Southern Europeans.

### Statistical Analysis

Associations were tested using Fisher's exact test under additive, dominant, and recessive models through PLINK. For the Northern European group, additional chi-square test were applied on alleles in order to estimate the genomic inflation factor, which is defined as the mean chi-square statistic from case/control association divided by the expected mean value of chi-square distribution.

### Power Simulation

The simulation conditions are listed in Table 10. For each combination of conditions, 1000 samplings (“simulations”) of minor alleles were performed in cases and controls (independently) from the binomial distribution with p at expected MAFs, which were inferred from the conditions. Power was defined as the proportion of simulations where p-values from Fisher's exact tests were smaller than cutoff values (10⁻⁶ or 5×10⁻⁸), assuming an additive model. The procedure was implemented in R (The R Project for Statistical Computing, version 2.6-2.9).

### CNV Analysis

The CNV calls were generated using the April 2009 version of PennCNV (Wang et al., 2007) software, applying the standard Hidden Markov model and population B allele frequency (BAF) for all SNPs and CNV probes included on the Illumina 1M chip. To ensure the accuracy of CNV calling, stringent sample and CNV filtering procedure was applied. We studied the relationship among the mean and standard deviation of Log R Ratio (normalized signal intensity from BeadStudio by Illumina) and the number of CNV calls, and found an excess of both all size and larger than 100 kb CNV calls in samples with LRR standard deviation greater than 0.23. We included all samples that had a LRR standard deviation <0.23, maximum number of total CNV calls <200, maximum number of 100 kb CNV call <20, BAF median>0.55 or <0.45, BAF drift >0.002 or WF>0.04 or <−0.04. Additionally, to ensure high-confidence CNVs, we excluded individual CNVs if:

1. PennCNV-generated confidence score <10;
2. calls based on fewer than 10 SNPs/CNV probes; and
3. spanned within 1 Mb from centromeres or telemeres.

Burden and common copy number variants association analysis was performed. Any copy number variants that were present in at least three subjects was considered to be common (Need et al., 2009). Associations were tested using two tails permuted (100000 times)

Fisher exact analysis using PLINK software, by considering duplications and deletions separately (Purcell et al., 2007). Singleton CNVs larger than 100 kb (Walsh et al., 2008) were also investigated to find evidence for individual predisposition to SJS/TEN. For this analysis, a all CNVs that had coverage greater than 20 genetic markers/CNVs were excluded. All analyses were performed on the North European samples that passed the genotyped quality control checks, excluding Italian cases since they did not have an ethnically matched control group.

### REFERENCES

Sambrook et al., Molecular Cloning, Cold Spring Harbor Laboratory Press, Cold Spring Harbor, N.Y., 1989.
Innis et al., Proc. Natl. Acad. Sci. USA, 85(24): 9436-9449, 1988.
Guilfoyle et al., Nucleic Acids Research, 25: 1854-1858, 1997.
Walker et al., Proc. Natl. Acad. Sci. USA, 89: 392-396, 1992.
Kwoh et al., Proc. Natl. Acad. Sci. USA, 86: 1173, 1989.
Frohman, PCR Protocols: A Guide to Methods and Applications, Academic Press, N.Y., 1990.
Ohara et al., Proc. Natl. Acad. Sci. USA, 86: 5673-5677, 1989.
Shenton et al., Drug Hypersensitivity, Basel, Karger, 2007, 115-128.
Slentz-Kesler et al., Genomics, 47: 327-340, 1998.
Blumberg et al., Cell, 104(1): 9-19, 2001.
Johnson et al., Proc. Natl. Acad. Sci. USA, 77: 3715-9, 1980.
Pirmohamed et al., Pharmacogenomics, 8(12): 1661-1691, 2007.
Kazeema et al., High resolution HLA genotyping and severe cutaneous adverse reactions in lamotrigine-treated patients, 2009.
Purcell et al., Am J Hum Genet, 81(3): 559-575, 2007.
Nelson et al., Am J Hum Genet, 83(3): 347-358, 2008.
Wellcome Trust Case Control Consortium, Nature, 447(7145): 661-678, 2007.
Nelson et al., Pharmacogenomics J, 9(1): 23-33, 2009.
Fukata et al., Science, 313(5794): 1792-1795, 2006.
Bastuji-Garin et al., Arch Dermatol, 129(1): 92-96, 1993.
Novembre et al., Nature, 456(7218): 98-101, 2008.
Wang et al., Genome Research, 17(11): 1665-1674, 2007.
Need et al., PLoS Genetics, 5(2): e1000373, 2009.
Walsh et al., Science, 320(5875): 539-543, 2008.

TABLE 1

| | | Position (NCBI | | |
|---|---|---|---|---|
| SNP Name | Chromosome | Build 36) | p-value | Odds Ratio |
| rs13436754 | 5 | 157063657 | 4.06E−06 | 4.7 |
| rs4704894 | 5 | 157069516 | 2.05E−06 | 5.1 |
| rs7745600 | 6 | 53195494 | 8.77E−06 | 4.1 |
| rs2498930 | 6 | 117515788 | 6.73E−06 | 3.1 |

TABLE 2

| | | Position (NCBI | | |
|---|---|---|---|---|
| SNP Name | Chromosome | Build 36) | p-value | Odds Ratio |
| rs35541527 | 1 | 160001221 | 1.34E−07 | 4.502 |
| rs4532807 | 1 | 160123820 | 1.95E−07 | 4.41 |
| rs16834361 | 1 | 160216498 | 2.97E−06 | 4.136 |
| rs10917742 | 1 | 161631060 | 8.18E−06 | 2.992 |
| rs10799916 | 1 | 161633478 | 9.81E−06 | 2.961 |
| rs12119507 | 1 | 161635485 | 9.81E−06 | 2.961 |
| rs10494383 | 1 | 161646376 | 8.18E−06 | 2.992 |
| rs6427805 | 1 | 198449277 | 1.18E−07 | 3.458 |
| rs2153279 | 1 | 198530320 | 1.79E−07 | 2.936 |
| rs7527464 | 1 | 198535414 | 1.71E−07 | 2.942 |
| rs7648260 | 3 | 177780230 | 7.95E−06 | 5.566 |
| rs3869129 | 6 | 31518628 | 4.20E−06 | 2.648 |
| rs6919586 | 6 | 31529091 | 9.62E−06 | 2.866 |
| rs7758412 | 6 | 39981124 | 9.14E−06 | 4.611 |
| rs3800349 | 6 | 40010178 | 5.52E−06 | 4.787 |
| rs17137412 | 7 | 7767212 | 7.54E−07 | 3.165 |
| rs10964685 | 9 | 20746763 | 4.90E−06 | 4.498 |
| rs745546 | 10 | 121532305 | 5.97E−06 | 4.184 |
| rs2991010 | 13 | 37189011 | 1.08E−06 | 3.738 |
| rs2787456 | 14 | 83596060 | 5.00E−06 | 3.484 |
| rs17379472 | 16 | 56250999 | 6.73E−07 | 4.811 |
| rs9896932 | 17 | 77848326 | 3.45E−06 | 3.657 |
| rs9898788 | 17 | 77849717 | 3.45E−06 | 3.657 |
| rs3176835 | 17 | 77862035 | 4.31E−06 | 3.838 |
| rs7288894 | 22 | 24846051 | 2.02E−06 | 7.078 |
| rs5945905 | X | 102006998 | 8.72E−06 | 2.7 |

TABLE 3

| | | Position (NCBI | | |
|---|---|---|---|---|
| SNP Name | Chromosome | Build 36) | p-value | Odds Ratio |
| rs4532807 | 1 | 160123820 | 4.72E−09 | 4.516 |
| rs16834361 | 1 | 160216498 | 2.36E−07 | 4.228 |
| rs10917742 | 1 | 161631060 | 5.06E−06 | 2.886 |
| rs10799916 | 1 | 161633478 | 7.79E−06 | 2.83 |
| rs12119507 | 1 | 161635485 | 7.79E−06 | 2.83 |
| rs10494383 | 1 | 161646376 | 7.22E−06 | 2.841 |
| rs2153279 | 1 | 198530320 | 3.63E−07 | 2.702 |
| rs7527464 | 1 | 198535414 | 3.52E−07 | 2.708 |
| rs4665037 | 2 | 159759313 | 6.94E−07 | 4.135 |
| rs4442987 | 2 | 227913226 | 2.21E−07 | 5.047 |
| rs2403522 | 3 | 130960501 | 2.92E−06 | 3.338 |
| rs7622619 | 3 | 154323451 | 5.45E−06 | 3.532 |
| rs2126633 | 4 | 22066790 | 5.91E−06 | 4.447 |
| rs7654229 | 4 | 22223400 | 8.17E−06 | 2.618 |
| rs7687322 | 4 | 100233365 | 7.83E−06 | 3.885 |
| rs11945682 | 4 | 167851923 | 6.90E−06 | 2.363 |
| rs9293603 | 5 | 91634965 | 1.26E−07 | 3.931 |
| rs488083 | 5 | 176195755 | 8.44E−06 | 4.347 |
| rs9501106 | 6 | 31496088 | 6.15E−07 | 2.887 |
| rs9469003 | 6 | 31515807 | 6.45E−06 | 2.588 |
| rs7758412 | 6 | 39981124 | 8.85E−12 | 7.607 |
| rs11969769 | 6 | 39982853 | 1.22E−11 | 7.521 |
| rs3800349 | 6 | 40010178 | 1.57E−11 | 7.45 |
| rs4896168 | 6 | 136056012 | 1.72E−06 | 4.744 |
| rs10872569 | 6 | 144797934 | 8.83E−06 | 3.229 |
| rs7819401 | 8 | 88900831 | 9.45E−06 | 2.585 |
| rs10762971 | 10 | 54751285 | 6.04E−06 | 4.397 |
| rs3740049 | 10 | 69241342 | 8.08E−06 | 3.082 |
| rs12262099 | 10 | 85982370 | 1.88E−06 | 5.188 |
| rs7091672 | 10 | 120586411 | 5.17E−06 | 2.416 |
| rs16911493 | 11 | 23531603 | 7.50E−06 | 5.061 |
| rs10877182 | 12 | 57567009 | 5.93E−07 | 4.253 |
| rs10877183 | 12 | 57567095 | 4.32E−07 | 4.322 |
| rs2991010 | 13 | 37189011 | 1.29E−06 | 3.253 |
| rs2787456 | 14 | 83596060 | 5.96E−07 | 3.477 |
| rs16956471 | 15 | 29113362 | 5.44E−06 | 2.623 |
| rs16967283 | 15 | 36802928 | 3.93E−06 | 3.03 |
| rs12913269 | 15 | 90727709 | 5.30E−06 | 3.19 |
| rs8041550 | 15 | 90731081 | 3.43E−06 | 3.264 |
| rs6497456 | 16 | 20191533 | 1.81E−06 | 4.572 |
| rs8053762 | 16 | 56180023 | 2.53E−06 | 4.737 |
| rs12708990 | 16 | 56198053 | 6.99E−06 | 4.18 |
| rs7212298 | 17 | 10965967 | 5.08E−06 | 4.035 |
| rs1984722 | 17 | 64500261 | 1.41E−11 | 6.678 |
| rs7221250 | 17 | 64532121 | 9.33E−10 | 5.641 |
| rs9896932 | 17 | 77848326 | 2.34E−08 | 4.208 |
| rs9898788 | 17 | 77849717 | 7.06E−10 | 4.833 |
| rs3176835 | 17 | 77862035 | 9.07E−08 | 4.296 |
| rs11575031 | 17 | 77872938 | 3.06E−07 | 4.059 |
| rs7224284 | 17 | 77906874 | 7.51E−06 | 3.6 |

TABLE 4
SNPs strongly associated with SSR by
diverse drugs

| | | | | MAF in | | | |
|---|---|---|---|---|---|---|---|
| SNPs | chr | Position | Genes | controls | p-value | OR | GT in cases |
| rs4532807 | 1 | 160123820 | ATF6 | 0.039 | 5 × 10⁻⁹ | 5 | 2, 12, 38 |
| rs12629207 | 3 | 138328949 | IL20RB | 0.019 | 1 × 10⁻⁹ | 6 | 2, 7, 43 |
| rs11969769 | 6 | 39982853 | MOCS1 | 0.014 | 1 × 10⁻¹¹ | 8 | 0, 10, 42 |
| rs9971363 | 10 | 64233708 | ADO | 0.055 | 3 × 10⁻¹⁰ | 4.4 | 2, 17, 33 |
| rs1984722 | 17 | 64500261 | ABCA9 | 0.019 | 1.4 × 10⁻¹¹ | 7 | 0, 12, 40 |
| rs9898788 | 17 | 77849717 | CD7, SECTM1 | 0.036 | 7 × 10⁻¹⁰ | 5 | 1, 14, 37 |

TABLE 5

| | | | Position | | | | |
|---|---|---|---|---|---|---|---|
| | | | (NCBI | | Allelic | MAF in | Nearby |
| Cohort | SNP | Chromosome | build36) | p-value | OR | controls | gene |
| n-EU SSR | rs17137412 | 7 | 7767212 | 3 × 10⁻⁸ | 3.7 | 0.1 | RPA3 |
| s-EU SSR | rs10098474 | 8 | 9949027 | 4 × 10⁻⁸ | 6 | 0.17 | MSRA |
| Bactrim SSR | rs7573804 | 2 | 40635501 | 2.7 × 10⁻⁸ | 8.5 | 0.06 | SLC8A1 |
| | rs4853428 | 2 | 79077410 | 6.9 × 10⁻⁸ | 7 | 0.16 | REG3G |
| | rs10492614 | 13 | 91081333 | 9.9 × 10⁻⁹ | 8.8 | 0.05 | GPC5 |
| Lamotrigine | rs12019361 | 7 | 87411583 | 3 × 10⁻⁸ | 13 | 0.06 | ADAM22 |
| SSR | | | | | | | |

TABLE 6
Causal drug summary of SJS/TEN collections

Supposed origin

| Drugs | nEU | sEU | cEU | eEU | Total |
|---|---|---|---|---|---|
| Cotrimoxazole | 11 | — | 1 | — | 12 |
| Lamotrigine | 7 | — | 2 | — | 9 |
| Amoxicillin | 3 | 5 | — | — | 8 |
| Phenytoin | 7 | — | — | 1 | 8 |
| Moxifloxacin | 2 | — | — | 1 | 3 |
| Carbamazepine | 3 | — | — | — | 3 |
| Allopurinol | 1 | 2 | — | — | 3 |
| Clarithromycin | 1 | 2 | — | — | 3 |
| Others | 20 | 13 | 1 | — | 34 |

Six subjects experienced SJS/TEN due to more than one causal drug.
nEU = North Europeans,
sEU = South Europeans,
cEU = Central Europeans,
eEU = Eastern Europeans

TABLE 7
Demographic and clinical characteristics of the enrolled patients summarized by cohort

| | Overall | % | European cohort | | | Diagnosis | | |
|---|---|---|---|---|---|---|---|---|
| collection | enrolled patients | Female | cases | controls | Case/Control ratio | SJS | Overlap | TEN |
| PGX40001 | 206 | 0.67 | 48 | 107 | 2.2 | 23 | 22 | 3 |
| LAM30004 | 69 | 0.51 | 5 | 51 | 10.20 | 5 | — | — |
| Italian Collection | 19 | 0.42 | 19 | — | case only | 14 | 3 | 2 |
| TSI | 88 | | — | 88 | controls only | — | — | — |
| WTCCC2 | 4500 | | — | 4251 | controls only | — | — | — |
| POPRES | 648 | | — | 648 | controls only | — | — | — |

TABLE 8
Top Associated SNPs

| | | | | Overall cases (72 vs 461) | | | | nEU (46 vs 4251) | | | |
|---|---|---|---|---|---|---|---|---|---|---|---|
| Marker | | | | logistic regression | | | | Fisher Exact test | | | |
| | | | Closest | MAF | | | OR | MAF | | | OR |
| Chr | SNP | type | gene | Ca | Co | P value | (95 CI) | Ca | Co | P value | (95 CI) |
| 6 | rs981946 | INTRON | SLC22A23 | 0.46 | 0.35 | 8.86E−06 | 2.3 | 0.52 | 0.36 | 0.00207 | 1.9 |
| | | IC | | | | | (1.6-3.4) | | | | (1.2-2.9) |
| 6 | rs1079284 | INTRON | SLC22A23 | 0.46 | 0.35 | 9.27E−06 | 2.3 | 0.52 | 0.36 | 0.002065 | 1.9 |
| | | IC | | | | | (1.6-3.4) | | | | (1.2-2.9 |
| 7 | rs17137412 | INTRON | RPA3/ | 0.24 | 0.12 | 0.000107 | 2.4 | 0.34 | 0.11 | 1.25E−08 | 4.0 |
| | | IC | GLCCI1 | | | | (1.5-3.7) | | | | (2.5-6.2) |
| 7 | rs12019361 | INTRON | ADAM22 | 0.14 | 0.07 | 0.004162 | 2.2 | 0.13 | 0.07 | 0.01879 | 2.1 |
| | | IC | | | | | (1.2-3.8) | | | | 1.1-3.9) |
| 11 | rs2448001 | INTRON | LGR4 | 0.49 | 0.31 | 5.16E−06 | 2.4 | 0.48 | 0.35 | 0.01567 | 1.6 |
| | | IC | | | | | (1.6-3.5) | | | | (1.1-2.5) |
| 11 | rs2472632 | INTRON | LGR4 | 0.49 | 0.31 | 5.87E−06 | 2.4 | 0.48 | 0.35 | 0.01592 | 1.6 |
| | | IC | | | | | (1.6-3.5) | | | | (1.1-2.5) |
| 12 | rs220549 | INTRON | GRIN2B | 0.49 | 0.43 | 0.1948 | 1.2 | 0.51 | 0.43 | 0.112 | 1.4 |
| | | IC | | | | | (0.8-1.8) | | | | (0.9-2.1) |
| 20 | rs6016348 | INTERGENIC | MAFB | 0.28 | 0.12 | 1.39E−06 | 2.9 | 0.29 | 0.13 | 4.15E−05 | 2.7 |
| | | | | | | | (1.9-4.5) | | | | (1.7-4.3) |
| 20 | rs6016358 | INTERGENIC | MAFB | 0.22 | 0.08 | 8.16E−06 | 2.9 | 0.24 | 0.09 | 1.27E−05 | 3.2 |
| | | | | | | | (1.8-4.7) | | | | (2.0-5.3) |

| | | | | sEU cohort (21 vs 97) | | | |
|---|---|---|---|---|---|---|---|
| Marker | | | | Logistic regression | | | |
| | | | Closest | MAF | | | OR |
| Chr | SNP | type | gene | Ca | Co | P value | (95 CI) |
| 6 | rs981946 | INTRON | SLC22A23 | 0.43 | 0.31 | 0.006278 | 2.9 |
| | | IC | | | | | (1.3-6.2) |
| 6 | rs1079284 | INTRON | SLC22A23 | 0.43 | 0.31 | 0.006278 | 2.9 |
| | | IC | | | | | (1.3-6.2) |
| 7 | rs17137412 | INTRON | RPA3/ | 0.07 | 0.08 | 0.8668 | 0.9 |
| | | IC | GLCCI1 | | | | (0.2-3.1) |
| 7 | rs12019361 | INTRON | ADAM22 | 0.12 | 0.08 | 0.3624 | 1.6 |
| | | IC | | | | | (0.5-5.2) |
| 11 | rs2448001 | INTRON | LGR4 | 0.43 | 0.31 | 0.004616 | 3.6 |
| | | IC | | | | | (1.4-9.0) |
| 11 | rs2472632 | INTRON | LGR4 | 0.43 | 0.31 | 0.004616 | 3.6 |
| | | IC | | | | | (1.4-9.0) |
| 12 | rs220549 | INTRON | GRIN2B | 0.45 | 0.43 | 0.6679 | 1.1 |
| | | IC | | | | | (0.5-2.3) |
| 20 | rs6016348 | INTERGENIC | MAFB | 0.26 | 0.13 | 0.02211 | 3.1 |
| | | | | | | | (1.1-8.3) |
| 20 | rs6016358 | INTERGENIC | MAFB | 0.19 | 0.10 | 0.07858 | 2.4 |
| | | | | | | | (0.9-6.5) |

Ca = cases;
Co = controls

TABLE 9
CNV analysis result

| | | diagnosis | | | | | | | |
|---|---|---|---|---|---|---|---|---|---|
| | | of | CNV | | | Start | End | length | |
| subject | collection | epilepsy | type | genotype | chr | Position | Position | (bp) | involved genes |
| case | PGX40001 | no | DUP | het | 1 | 103776619 | 106583135 | 2806517 | RNPC3, AMY2B, |
| | | | | | | | | | AMY2A, AMY1A, |
| | | | | | | | | | AMY1C, AMY1B |
| control | LAM30004 | yes | DUP | het | 1 | 144933825 | 145848182 | 914358 | PRKAB2, PDIA3P, |
| | | | | | | | | | FMO5, CHD1L, |
| | | | | | | | | | BCL9, ACP6, |
| | | | | | | | | | GJA5, GJA8, |
| | | | | | | | | | GPR89B |
| control | PGX40001 | no | DUP | het | 1 | 91197164 | 92008636 | 811473 | ZNF644, |
| | | | | | | | | | HFM1, CDC7, |
| | | | | | | | | | TGFBR3 |
| case | LAM30004 | yes | DUP | het | 2 | 124743745 | 125785851 | 1042107 | CNTNAP5 |
| control | PGX40001 | no | DUP | het | 2 | 32483938 | 33184723 | 700786 | BIRC6, TTC27, |
| | | | | | | | | | LTBP1 |
| control | PGX40001 | no | DUP | het | 3 | 2373746 | 2996663 | 622918 | CNTN4 |
| case | PGX40002 | yes | DEL | het | 7 | 124892028 | 125395114 | 503087 | GRM8 |
| control | PGX40001 | no | DUP | het | 12 | 75994250 | 76631650 | 637401 | between E2F7 and |
| | | | | | | | | | NAV3 |
| case | PGX40001 | yes | DEL | het | 13 | 111553204 | 114114639 | 2561436 | SOX1, AK055145, |
| | | | | | | | | | C13orf28, |
| | | | | | | | | | TUBGCP3, |
| | | | | | | | | | C13orf35, |
| | | | | | | | | | ATP11A, MCF2L, |
| | | | | | | | | | F7, F10, PROZ, |
| | | | | | | | | | PCID2, CUL4A, |
| | | | | | | | | | LAMP1, GRTP1, |
| | | | | | | | | | ADPRHL1, |
| | | | | | | | | | DCUN1D2, |
| | | | | | | | | | TMCO3, TFDP1, |
| | | | | | | | | | ATP4B, GRK1, |
| | | | | | | | | | BC034570, GAS6, |
| | | | | | | | | | DQ866763, |
| | | | | | | | | | FAM70B, RASA3, |
| | | | | | | | | | CDC16 |
| control | PGX40001 | no | DUP | het | 15 | 51677654 | 52244046 | 566393 | WDR72 |
| case | PGX40001 | yes | DUP | het | 20 | 45902724 | 46559948 | 657225 | between SULF2 |
| | | | | | | | | | and PREX1 |

TABLE 10
Conditions of power simulation

| Parameters | Values |
|---|---|
| Number of cases | 9, 12, 15, 18, 21, 24, 30, 36, 42, 49, 56, 63, 70 |
| Number of controls | 100, 143, 1600, 4900, 6400 |
| Odds Ratio (OR) | 2, 2.5, . . . 50 (step: 0.5) |
| MAF in the population | 0.1, . . . , 0.5 (step: 0.01) |
| (MAF_control) | |
| Prevalence | 0.001 |

1. A method of identifying a subject afflicted with, or at risk of developing, Serious Skin Rash (SSR) comprising:
(a) obtaining a nucleic-acid containing sample from the subject; and
(b) analyzing the sample to detect the presence of at least one genetic marker, or an equivalent to at least one genetic marker, selected from those in Tables 1, 2, 3, 4, 5 and 8,
wherein the presence of at least genetic marker, or an equivalent to at least one genetic marker, from Tables 1, 2, 3, 4, 5 and 8 in the sample indicates that the subject is afflicted with, or at risk of developing, SSR.

2. The method of claim 1, wherein the at least one genetic marker is a single nucleotide polymorphism (SNP), an allele, a microsatellite, a haplotype, a copy number variant (CNV), an insertion, or a deletion.

3. The method of claim 2, wherein the genetic marker is an SNP selected from one of rs4532807, rs12629207, rs11969769, rs9971363, rs1984722, rs9898788, rs7758412, rs17137412, rs10098474, rs12019361, rs981946, rs1079284, rs2448001, rs2472632, rs220549, rs6016348, and rs6016358.

4. The method of claim 1, wherein the analysis of the sample comprises nucleic acid amplification.

5. The method of claim 4, wherein the amplification comprises PCR.

6. The method of claim 1, wherein the analysis of the sample comprises primer extension.

7. The method of claim 1, wherein the analysis of the sample comprises restriction digestion.

8. The method of claim 1, wherein the analysis of the sample comprises DNA sequencing.

9. The method of claim 1, wherein the analysis of the sample comprises SNP specific oligonucleotide hybridization.

10. The method of claim 1, wherein the analysis of the sample comprises a DNAse protection assay.

11. The method of claim 1, wherein the analysis of the sample comprises mass spectrometry.

12. The method of claim 1, wherein the sample is selected from one of serum, sputum, saliva, mucosal scraping, tissue biopsy, lacrimal secretion, semen, or sweat.

13. The method of claim 1, further comprising treating the subject for SSR based on the results of step (b).

14. The method of claim 1, further comprising taking a clinical history of the subject.

15. The method of claim 1, wherein the SSR is caused by one or more of nonsteroidal anti-inflammatory agents (NSAIDs), sulfonamides, anticonvulsants, allopurinol, and antimalarials.

16. A method of identifying a therapeutic agent for the treatment of SSR, comprising:
(a) contacting cells expressing at least one genetic marker from Tables 1, 2, 3, 4, 5 and 8 with a putative therapeutic agent; and
(b) comparing expression of the cells prior to contact with the putative therapeutic agent to expression of the cells after contact with the putative therapeutic agent;
wherein a decrease in expression of the cells after contact with the putative therapeutic agent identifies the agent as an agent for the treatment of SSR.
